(19) United States
(12) Patent Application Publication
(10) Pub. No.: US 2003/0190618 A1
(43) Pub. Date: Oct. 9, 2003

(54) Method for generating five prime biased tandem tag libraries of cDNAs

(76) Inventors: Babru Samal, N. Potomac, MD (US); Yuan Li, Rockville, MD (US); Leandro C. Hermida, Germantown, MD (US); Nancy L. Hoppa, Westminster, MD (US); Karl K. Johe, Potomac, MD (US)

Correspondence Address: BELL, BOYD & LLOYD LLC, P.O. Box 1135, Chicago, IL, 60690-1135, US

(21) Appl. No.: 10/092,885

(22) Filed: Mar. 6, 2002

(51) Int. Cl. C12Q 1/68; C12P 19/34

(52) U.S. Cl. 435/6; 435/91.2

(57) ABSTRACT

A method for generating five prime biased tandem tag libraries of cDNAs is revealed. The method allows generation of partial sequences consisting of a minimal length of expressed cDNA sequences of at least 20 bases from biological samples to rapidly identify novel expressed transcripts.

### BACKGROUND OF THE INVENTION

1. Field of the Invention

The sequences of whole genomes from several organisms have now been elucidated and are available as searchable databases. This enables rapid identification of full-length messenger RNAs (mRNAs) expressed in a biological sample once a partial sequence is known. The method described here allows generation of such partial sequences consisting of a minimal length of expressed cDNA sequences of at least 20 bases from biological samples to rapidly identify novel expressed transcripts.

2. Description of the Related Art

In order to obtain a comprehensive collection of all human genes that are expressed, many millions of cDNA molecules must be sequenced, which is quite costly and laborious. Since the availability of the human genome sequence, much of the coding sequence of a gene can now be inferred once a short physical sequence is obtained. Hence, sequencing only a short stretch of cDNAs should be sufficient in theory to identify all genes expressed in a biological sample. The Expressed Sequence Tag (EST) method purports to achieve this by generating for sequencing relatively short cDNA fragments from 3′ ends. However, the EST method still utilizes one cDNA per clone, which means one sequencing reaction yields one cDNA sequence.

An effective way to improve this yield so that each plasmid and each sequencing reaction yields many cDNA sequences is to “glue” together short cDNA fragments from end to end. The Serial Gene Expression Analysis (SAGE) method effectively utilizes such a concatenation procedure. The SAGE method, however, has two key shortcomings. One is that all of the tags are generated from a defined 3′ end of a cDNA. Mammalian genes contain long untranslated sequences at their 3′ ends, which make the determination of coding sequence by gene prediction algorithms difficult and unreliable. The second limitation is that the SAGE tags are typically only 14 bases long, which are too short to yield uniquely matching sequences from the genomic database. A minimum of 20 bases is needed to identify a uniquely matching gene from a mammalian genomic database at 80% of the time.

Thus, the most important prerequisite for obtaining expressed sequence tags to rapidly and uniquely identify coding sequences from a messenger RNA pool is to obtain expressed sequence tags of 20 bases or longer from the 5′ end of a coding region. Such tags then can be used as a forward PCR primer to easily amplify, sequence, and clone each gene uniquely. There is presently no method, which predictably generates 5′ cDNA fragments of 20-40 bases. The method described here generates one or more short tags at or near the 5′ end of each gene transcript in tandem or in cluster so that when they are aligned against genomic sequences they together uniquely identify a contiguous expressed sequence of 20 bases or greater.

### SUMMARY OF THE INVENTION

The present application discloses a method for generating five prime biased tandem tag libraries of cDNAs. The method comprises the steps of isolating a sample of mRNAs; synthesizing double-stranded cDNAs from the mRNAs; blunt-ending the double-stranded cDNAs; attaching an adapter molecule to the blunt ends of the double stranded cDNAs to form a complex, where the adapter molecule is a double stranded, synthetic oligonucleotide comprising a recognition site for a type IIS restriction enzyme, a cloning site for releasing tags to a cloning vector, and a PCR primer site; digesting the complex with a type IIS restriction enzyme to form released tags; separating the released tags from the double-stranded cDNAs; amplifying the released tags to form amplified tags; isolating the amplified tags; concatenating the amplified tags to form concatenated tags; amplifying the concatenated tags; and isolating the concatenated tags.

In a preferred embodiment, the type IIS restriction enzyme is selected from the group consisting of Ear I, Sap I, Alw I, Bmr I, Bsa I, BsmA I, BsmB I, Mly I, Ple I, Bbs I, BciV I, Fau I, Mnl I, Aar I, BfuA I, BspM I, Hph I, Mbo II, SspD5 I, Sth132 I, SfaN I, BseR I, BspCN I, Hga I, AceIII, Eci I, TaqII, Tth111II, Bbv I, RleAI, BcefI, Fok I, BceA I, BsmF I, StsI, Bce83I, BpmI, Bsg I, Eco57I, Eco57MI, and MmeI. In a more preferred embodiment, the type IIS restriction enzyme is BpmI.

In another preferred embodiment, the mRNAs are from a mammal. In a more preferred embodiment, the mRNAs are from a human.

In other preferred embodiments, the released tags are comprised of 50 nucleotides or less; the released tags are comprised of 36 nucleotides or less; the released tags are comprised of 32 nucleotides or less. In a more preferred embodiment, the released tags are comprised of at least 20 nucleotides.

In yet another preferred embodiment, the method further comprises sequencing the isolated concatenated tags to obtain a nucleotide sequence and comparing the nucleotide sequence to a known nucleotide sequence.

The present application also discloses a method for generating five prime biased tandem tag libraries of cDNAs, comprising the steps of isolating a sample of mRNAs; synthesizing double-stranded cDNAs from the mRNAs; blunt-ending the double-stranded cDNAs; attaching a first adapter molecule to the blunt ends of the double stranded cDNAs to form a first complex, where the first adapter molecule is a double stranded, synthetic oligonucleotide comprises a recognition site for a type IIS restriction enzyme, a cloning site for releasing tags to a cloning vector, and a PCR primer site; digesting the first complex with a type IIS restriction enzyme to form first released tags; separating the first released tags from the double-stranded cDNAs and attaching a second adapter molecule to the double-stranded cDNAs to form a second complex; amplifying the first released tags to form first amplified tags; isolating the first amplified tags; concatenating the first amplified tags to form first concatenated tags; amplifying the first concatenated tags; isolating the first concatenated tags; digesting the second complex with a type IIS restriction enzyme to form second released tags; separating the second released tags from the double-stranded cDNAs; amplifying the second released tags to form second amplified tags; isolating the second amplified tags; concatenating the second amplified tags to form second concatenated tags; amplifying the second concatenated tags; and isolating the second concatenated tags.

In a preferred embodiment, the type IIS restriction enzyme is selected from the group consisting of Ear I, Sap I, Alw I, Bmr I, Bsa I, BsmA I, BsmB I, Mly I, Ple I, Bbs I, BciV I, Fau I, Mnl I, Aar I, BfuA I, BspM I, Hph I, Mbo II, SspD5 I, Sth132 I, SfaN I, BseR I, BspCN I, Hga I, AceIII, Eci I, TaqII, Tth111II, Bbv I, RleAI, BcefI, Fok I, BceA I, BsmF I, StsI, Bce83I, BpmI, Bsg I, Eco57I, Eco57MI, and MmeI. In a more preferred embodiment, the type IIS restriction enzyme is BpmI.

In another preferred embodiment, the mRNAs are from a mammal. In a more preferred embodiment, the mRNAs are from a human.

In other preferred embodiments, the released tags are comprised of 50 nucleotides or less; the released tags are comprised of 36 nucleotides or less; the released tags are comprised of 32 nucleotides or less. In a more preferred embodiment, the released tags are comprised of at least 20 nucleotides.

In yet another preferred embodiment, the method further comprises sequencing the isolated concatenated tags to obtain a nucleotide sequence and comparing the nucleotide sequence to a known nucleotide sequence.

### BRIEF DESCRIPTION OF THE DRAWINGS

FIGS. 1A, 1B and 1C show a flow chart of an embodiment of the present method for generating five primed biased tandem tag libraries of cDNAs.

### DETAILED DESCRIPTION

A. Brief Description of the Method

1. The first and second strand cDNA synthesis is carried out according the standard procedure. In a preferred embodiment, the first strand synthesis is carried out with olido-dT 3′ primer covalently linked to magnetic beads according to the manufacturer's protocol (Dynal Inc.).

2. The 5′ ends of the ds-cDNAs are flushed using T4 DNA polymerase in the presence of dNTP, followed by the ligation of a double stranded adaptor. The adaptor can be of any sequence but contains the recognition sequence for a type IIS restriction enzyme that cleaves double stranded DNA substrates at some length downstream of the recognition site. In a preferred embodiment, the recognition sequence for a type IIS enzyme, Bpm I (also known as Gsu I) was placed at the 3′ end of the adaptor so that the nucleotide sequence immediately following the Bpm I site is from cDNAs. In addition, optionally, the recognition site for a rare six cutter such as the Mlu I enzyme can also be incorporated into the adaptor at just upstream of the Bpm I site to be utilized at a later step. The remaining adaptor sequence serves as the forward primer site for a subsequent PCR amplification step.

3. The ligated adaptor-cDNAs are purified and then digested with Bpm I to release the 16 bp cDNA tags plus the adaptor. The rest of the cDNAs remain bound to the magnetic beads and saved.

4. The adaptor-tag fragments are recovered by separating away the magnetic beads. They are ligated with a second adaptor of an arbitrary sequence but containing the same Mlu I site at the 5′ end of the adaptor. These two adaptors also facilitate PCR amplification of the internal 16 bp cDNA tags.

5. PCR amplification is carried out according to the standard procedure using the forward and reverse primers, which contain the sequences of the two adaptors respectively. The product is purified and ligated to a PCR cloning vector followed up by the transformation of competent bacteria. 6. Plasmid harboring colonies are drug-selected. The plasmid DNA is purified and digested with Mlu I. The released tags plus the restriction sites (28 bp) are isolated and ligated to form concatamers. The concatmers of appropriate size, typically 0.5 Kb -1.5 Kb, are fractionated by agarose gel-electrophoresis and then ligated into a Mlu I cut vector. After cloning, the 16 bp cDNA tags are elucidated by sequencing the concatemers.

7. The remaining cDNAs bound to the magnetic beads from the step 3 are then processed again through steps 2-6 to generate the second 16 bp tag from each cDNA. Thus, after the two rounds, two tandem tags from the 5′ end of each expressed transcript are generated, which, when aligned against the genomic sequence, generate 32 bases of combined sequence.

8. Steps 2-6 can be repeated several times as necessary.

B. More Detailed Description of the Method

Step 1: cDNA Synthesis

Total RNA was isolated from the HK 532 Cortical Cell Line using the Qiagen total RNA isolation kit (Qiagen, Inc., Valencia, Calif.). Briefly, the cells were lysed in a lysis buffer followed by binding of the RNA to the Qiagen solid matrix, from which the RNA was eluted, precipitated and kept at −20° C. overnight.

Messenger RNA (mRNA), typically of 200 ng, was incubated with Dynal beads (Dynal, Inc., Lake Success, N.Y.) containing oligo(dT) to attach the polyadenylated RNA which was converted into cDNA using the Superscript II cDNA synthesis kit (GIBCO Life Technologies, Gaithersburg, Md.) according to the manufacturer's directions.

Step 2: Adaptor Ligation

After the second strand synthesis, the 5′ ends of the double stranded-cDNA (ds-cDNA) were flushed using T4 DNA polymerase. Oligonucleotide adaptors were created by mixing equimolar amount of each of two synthetic oligonucleotides

sense strand:

GCAGTGGTATCAACGCAGAGTCCAGTGTGGTGGACGCGTCTGGAG (SEQ ID NO: 1)

antisense strand:

ₚCTCCAGACGCGTCCACCACACTGGACTCTGCGTTGATACCAC (SEQ ID NO: 2)

in deionized water, heating them to 95° C., and allowing them to cool slowly to room temperature to form:

PCR primer site Mlul_BpmI

(SEQ ID NO:3)

5′GCAGTGGTATCAACGCAGAGTCCAGTGTGGTGGACGCGTCTGGAG
|||||||||||||||||||||||||||||||||||||||||
CACCATAGTTGCGTCTCAGGTCACACCACCTGCGCAGACCTCₚ

Adaptor DNA (500 pmoles) was added to the solid-phase cDNA in a total volume of 50 μl of 1× ligase buffer containing 25 U of T4 ligase (Gibco BRL). The reaction was incubated overnight at 16° C. followed by 10 min at 65° C. to inactivate the enzyme.

Step 3: Release and Recovery of the First Tag

Beads were again washed extensively in wash buffer (5 mM TrisHCl, pH 8.0, 0.5 mM EDTA, 1M NaCl and 200 μg BSA/ml), followed by three washes in BpmI buffer, and resuspended in 50 μl of Bpm I buffer containing 50 U of Bpm I and incubated at 37° C. for 5 h with gentle rotation. The tag-containing supernatant was collected and the beads were washed twice with 100 μl of reaction buffer 3 (NEBL, Beverly, Mass.). The supernatant and washes were combined. The combined material was extracted with phenol:CIA. A half volume of 7.5 M ammonium acetate, or a one-third volume of 10 M ammonium acetate was added and DNA was precipitated with 2 volumes of ethanol in the presence of 4 μl of glycogen (20 mg/ml) per 300 μl of initial volume.

Step 4: Ligation of the 3′ Adaptor

A second, 16-fold degenerate adaptor molecule was prepared by annealing synthetic oligos as described above

sense strand:

ₚACGCGTGTCGACCTCGAGT (SEQ ID NO: 4);

antisense strand:

TCTAGACTCGAGGTCGACACGCGTNN (SEQ ID NO: 5)

to give the following oligodimer:

Mlu I PCR primer site

| | |
|---|---|
| Mlu I PCR Primer site | |
| ₚACGCGTCTCGACCTCGAGT | (SEQ ID NO:6) |
| \|\|\|\|\|\|\|\|\|\|\|\|\|\|\|\|\| | |
| NNTGCGCACAGCTGGAGCTCAGATCT | |

Five hundred pmol of adaptor were added to the tag DNA in a total volume of 50 μl of 1× ligase buffer containing 10U of T4 DNA ligase and incubated overnight at 16° C. The ligase was inactivated by incubation at 65° C. for 10 min.

Step 5: PCR Amplification of the Tags

PCR amplification of the tags was carried out using sense and antisense primers designed to match the two adaptor sequences.

The following primers were used:

forward

5′ TCTAGACTCGAGGTCGACACGC (SEQ ID NO: 7)

and reverse

5′ GCAGTGGTATCAACGCAGAGTCC (SEQ ID NO: 8)

Step 6: Tag Concatenation

The PCR product was electrophoresed on a polyacrylamide gel to isolate the 85 bp tag band. After phenol:CIA extraction and ethanol precipitation, the DNA was suspended in TE (pH 7.5). DNA was ligated with TA cloning vector (In Vitrogen, Inc, Carlsbad, Calif.). Transformation was carried out according to the protocol provided by the manufacturer.

Transformed E. coli cells were grown in 100 ml of ampicilin-containing Terrific Broth at 37° C., shaken at 300 rpm for 16 hr. Plasmid DNA preparation was carried out using Maxi kit (Qiagen Inc). About 750 μg DNA was obtained which was suspended in 500 μl of water.

The digestion of the purified plasmid DNA was carried out in a volume of 750 μl using 2 Units of Mlu I per μg of plasmid DNA for 4 hours. The resulting 28 bp tags were purified by electrophoresis on a 1.0% agarose gel in TAE buffer.

The 28 bp band was cut out of the gel, and eluted using a freeze-thaw technique. The DNA was extracted with phenol:CIA and ethanol precipitated in the presence of 4 μl glycogen and 100 μl of 10 M ammonium acetate per every 300 μl of sample. DNA was then resuspended in 16 μl water.

Concatemers were formed in a final volume of 20 μl using 1 μl of T4 DNA ligase (NEB, 400 units/μl). Concatemers were fractionated on an agarose gel isolating greater than 500 bp fragments. The fragments were purified using the Qiaex (Qiagen, Valencia, Calif.) protocol following the manufactures's instructions. The large molecular weight concatemers were then ligated into Mlu I-digested, alkaline phosphatase-treated, pBlueScript plasmid in which an Mlu I site had been engineered.

Results

The accuracy with which one can align a short cDNA sequence to the genomic sequence depends upon the length of the cDNA sequence. This is illustrated in TABLE 1 below. Using the NCBI Database of 47,584 known and hypothetical mRNAs, short expressed sequences (tags) from the 5′ end of mRNAs were extracted and aligned against the genomic database. The result clearly demonstrates that at least 20 bases and preferably 32 bases or more of a contiguous sequence of mRNA are required to obtain a unique genomic match and thereby to identify a coding region from a genomic database.

TABLE 1
Effect of Tag Length on Unique Genomic Hits

| TAG | |
|---|---|
| LENGTH | % TAGS WITH UNIQUE GENOMIC HIT |
| 14 | 5.76 |
| 16 | 37.56 |
| 18 | 74.47 |
| 20 | 84.56 |
| 32 | 89.44 |
| 36 | 90.07 |
| 40 | 90.61 |

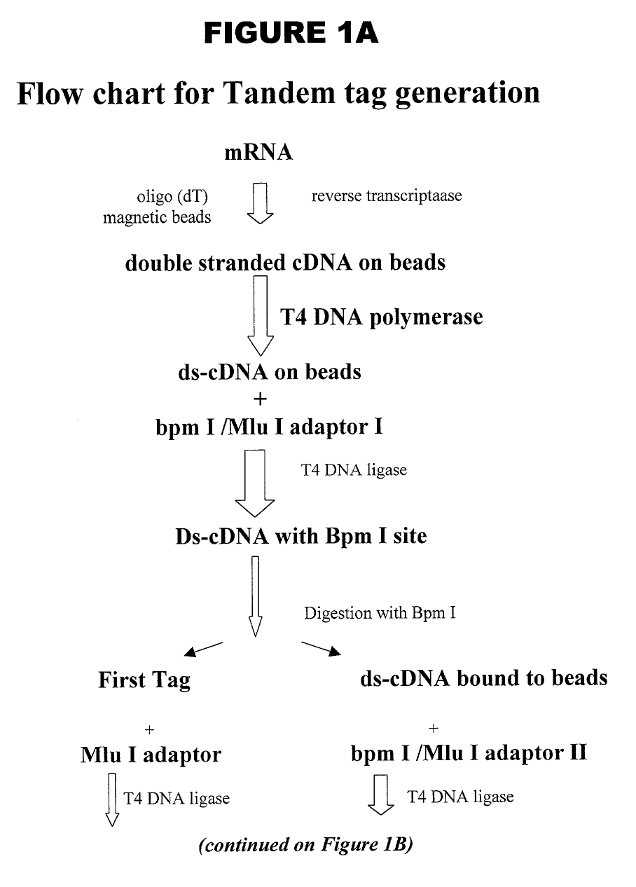

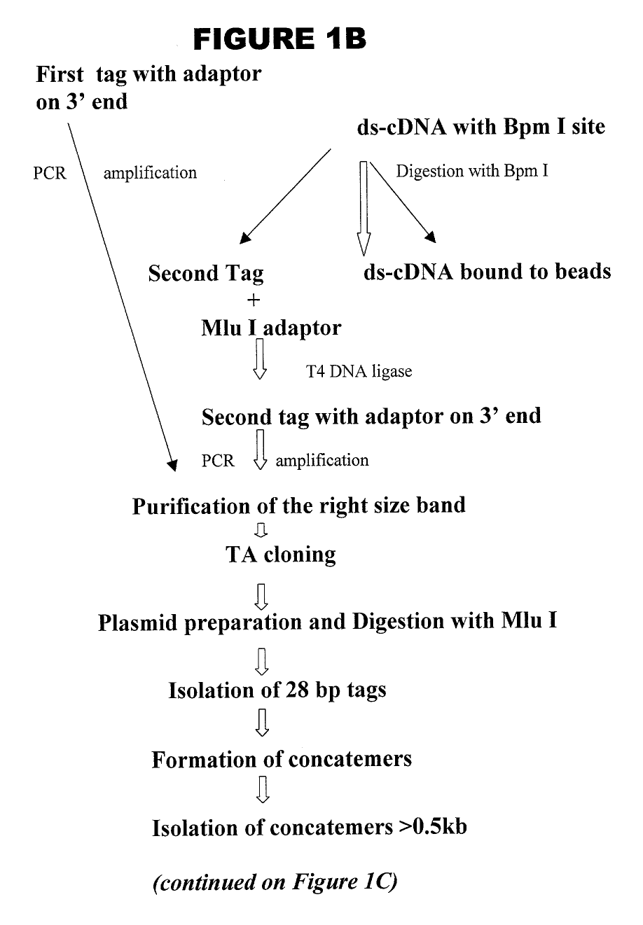

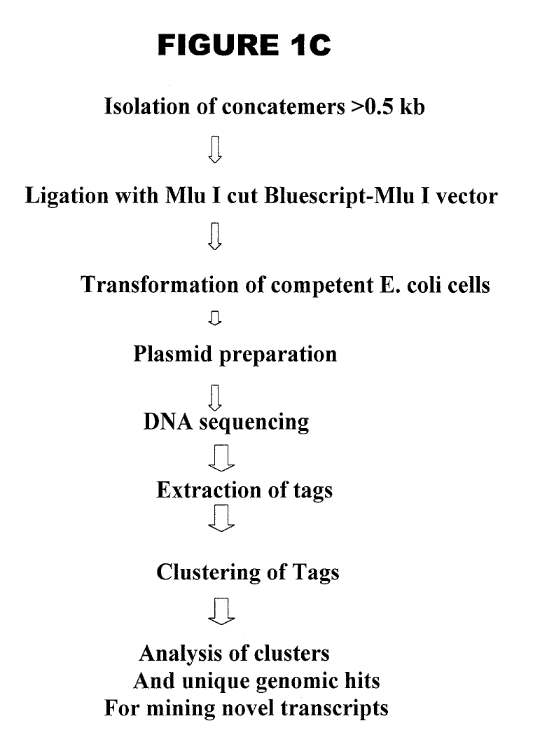

However, currently, there is no enzyme, which can reproducibly generate 20 bases or longer fragments of double stranded cDNAs. We have developed a method to generate such expressed fragments. By obtaining one or more successive shorter fragments (tags) of 10-20 bases, which can then be aligned against the genomic sequence, the method generates two tandem tags which, in effect, produces a long contiguous sequence of 20 bases or greater. As a preferred embodiment, we have used an enzyme, Bpm I, which generates 16 base pair tags each time and 32 base pair tandem tags when aligned. A schematic outline of the method is shown in FIGS. 1A, 1B and 1C.

As an example, a tandem tag library, i.e., two successive tag libraries from a single cDNA sample, was generated from the mRNA of a human cortical neural stem cell culture consisting of approximately 2×10⁷ cells. The resulting tag libraries were sequenced, aligned against the human genomic database, and pairs of tags, which align perfectly end to end on the genomic sequence were identified as tandem tags. Some of the tandem tags are shown in TABLE 2 and TABLE 3.

In TABLE 2, the two tandem 16-mer tags which uniquely and perfectly match known mRNA sequences are shown. The NCBI database of 47,584 known and hypothetical mRNAs was used as the template. In TABLE 3, the human genomic database was used first as the template to generate tandem tags. These were then compared to the mRNA database to verify whether the tandem tags indeed identified a coding region. These tandem tags are also found to be tandem within a known mRNA. BLAST of mRNA sequence to the human genome reveals that tandem genomic alignment was correct in each case.

TABLE 2
Examples of 16mer Tags Found to be Tandem within Known Transcripts

| | MATCHING mRNA | TANDEM TAG | |
|---|---|---|---|
| TAGS | ACCESSION NO. | SEQUENCE POS. | mRNA NAME/DESCRIPTION |
| GCGCGGTGTGGTGGCA | NM_001024.2 | 14 | Homo sapiens ribosomal protein S21 |
| (SEQ ID NO: 9)/ | | | (RPS21), mRNA |
| GCAGGCGCAGCCCAGC | | | |
| (SEQ ID NO: 10) | | | |
| GATAGATCGCCATCAT | NM_033022.1 | 24 | Homo sapiens ribosomal protein S24 |
| (SEQ ID NO: 11)/ | | | (RPS24), mRNA |
| GAACGACACCGTAACT | | | |
| (SEQ ID NO: 12) | | | |
| TAGATCGCCATCATGA | NM_033022.1 | 26 | Homo sapiens ribosomal protein S24 |
| (SEQ ID NO: 13)/ | | | (RPS24), mRNA |
| ACGACACCGTAACTAT | | | |
| (SEQ ID NO: 14) | | | |
| CTGCGGTGGAGCCGCC | NM_002954.2 | 23 | Homo sapiens ribosomal protein S27a |
| (SEQ ID NO: 15)/ | | | (RPS27A), mRNA |
| ACCAAAATGCAGATTT | | | |
| (SEQ ID NO: 16) | | | |
| GTGGAGCTGTCGCCAT | NM_000986.1 | 26 | Homo sapiens ribosomal protein L24 |
| (SEQ ID NO: 17)/ | | | (RPL24), mRNA |
| GAAGGTCGAGCTGTGC | | | |
| (SEQ ID NO: 18) | | | |
| GCCATCGTGGTGTGTT | NM_001000.1 | 3 | Homo sapiens ribosomal protein L39 |
| (SEQ ID NO: 19)/ | | | (RPL39), mRNA |
| CTTGACTCCGCTGCTC | | | |
| (SEQ ID NO: 20) | | | |
| CAGCACCATGGCGGTT | NM_001006.1 | 30 | Homo sapiens ribosomal protein S3A |
| (SEQ ID NO: 21)/ | | | (RPS3A), mRNA |
| GGCAAGAACAAGCGCC | | | |
| (SEQ ID NO: 22) | | | |
| CTTGAACCTGGGAGGC | XM_040175.1 | 2779 | Homo sapiens NADH dehydrogenase |
| (SEQ ID NO: 23)/ | | | (ubiquinone) Fe-S protein 8 (23 kD) |
| GGAGGTTGCAGTGAAC | | | (NADH-coenzyme Q reductase) (NDUFS8), |
| (SEQ ID NO: 24) | | | mRNA |
| CTTGAACCCAGGAGGT | XM_035578.1 | 1853 | Homo sapiens similar to X-like 1 protein |
| (SEQ ID NO: 25)/ | | | (LOC91023), mRNA |
| GGAGGTTGCAGTGATC | | | |
| (SEQ ID NO: 26) | | | |
| GTGTGTGTGTGTGTGT | NM_016352.1 | 2513 | Homo sapiens carboxypeptidase A3 |
| (SEQ ID NO: 27)/ | | | (LOC51200), mRNA |
| GTTTGTGTGTGTGTGT | | | |
| (SEQ ID NO: 28) | | | |

TABLE 3
Examples of Tags with Tandem Genome Alignment and Tandem mRNA Alignment; mRNA
CDS found at location of Tandem Genome Alignment

| | GENOME LOCATION | MRNA LOCATION | |
|---|---|---|---|
| | OF | OF TANDEM | BLAST RESULTS OF MRNA |
| TAGS | TANDEM MATCH | MATCH | TO GENOME ALIGNMENT |
| CTGCGGTGGAGCCGCC | NT_007741.6 | NM_002954.2 @ | NT_007741.6 MINUS |
| (SEQ ID NO: 29)/ | MINUS strand @ | 23 (RPS27a) | strand 1,292,043- |
| ACCAAAATGCAGATTT | 1,292,008 | | 1,291,507 |
| (SEQ ID NO: 30) | | | |
| GTGGAGCTGTCGCCAT | NT_007592.6 | NM_000986.1 @ | NT_007592.6 MINUS |
| (SEQ ID NO: 31)/ | MINUS strand @ | 26 (RPL24) | strand 1,993,292- |
| GAAGGTCGAGCTGTGC | 1,993,254 | | 1,992,861 |
| (SEQ ID NO: 32) | | | |
| GCCATCGTGGTGTGTT | NT_007236.6 | NM_001000.1 @ | NT_007236.6 MINUS |
| (SEQ ID NO: 33)/ | MINUS strand @ | 3 (RPL39) | strand 3,673,641- |
| CTTGACTCCGCTGCTC | 3,673,626 | | 3,673,273 |
| (SEQ ID NO: 34) | | | |
| CAGCACCATGGCGGTT | NT_007816.6 | NM_001006.1 @ | NT_007816.6 MINUS |
| (SEQ ID NO: 35)/ | MINUS strand @ | 30 (RPS3A) | strand 2,168,129- |
| GGCAAGAACAAGCGCC | 2,168,098 and | | 2,167,273 |
| (SEQ ID NO: 36) | 2,229,441 | | NT_007816.6 MINUS |
| | | | strand 2,229,472- |
| | | | 2,228,616 |
| CTTGAACCCAGGAGGT | NT_010204.6 PLUS | XM_035578.1 @ | NT_010204.6 PLUS |
| (SEQ ID NO: 37)/ | strand @ | 1853 (X-like 1 | strand 1,472,273- |
| GGAGGTTGCAGTGATC | 1,527,899 | protein) | 1,527,982 |
| (SEQ ID NO: 38) | | | |
| CTTGAACCCAGGAGGT | NT_029281.1 PLUS | XM_043233.1 @ | NT_029281.1 PLUS |
| (SEQ ID NO: 39)/ | strand @ 84,817 | 875 (AK022192) | strand 83,943- |
| TGCAGTGAGCCAAGAT | | | 86,079 |
| (SEQ ID NO: 40) | | | |

To further test the efficiency of the tandem tags to identify coding regions within the human genome, 400 random 16-mers from the first tag library and 400 random 16-mers from the second tag library were selected. Tandem tags were identified from the genomic database. As shown in TABLE 4, the 32-mer tandem tags were vastly more efficient in zeroing on the uniquely matching coding region of the human genome than the individual 16-mer tags.

TABLE 4
Tandem vs. Non-tandem Efficiency

| | GENOME |
|---|---|
| TAGS | MATCHES |
| GCACTTTGGGAGGCCGGCTCACGCCTGTAATC | 1 |
| (SEQ IN NO:41) | |
| GCACTTTGGGAGGCCG | 157, 201 |
| (SEQ ID NO:42) | |
| GCTCACGCCTGTAATC | 170, 672 |
| (SEQ ID NO:43) | |
| CACGCCCGTAATCCCAAGCACTTTGGGAGGCT | 1 |
| (SEQ ID NO:44) | |
| CACGCCCGTAATCCCA | 1, 337 |
| (SEQ ID NO:45) | |
| AGCACTTTGGGAGGCT | 132, 561 |
| (SEQ ID NO:46) | |
| AGCACTTTGGGAGGCTGAGATCGAGACCATCC | 2 |
| (SEQ ID NO:47) | |
| AGCACTTTGGGAGGCT | 132, 561 |
| (SEQ ID NO:48) | |
| GAGATCGAGACCATCC | 66, 177 |
| (SEQ ID NO:49) | |
| GCTTGAACCTGGGAGGGGAGGTTGCAGTGAGC | 10 |
| (SEQ ID NO:50) | |
| GCTTGAACCTGGGAGG | 62, 132 |
| (SEQ ID NO:51) | |
| GGAGGTTGCAGTGAGC | 162, 173 |
| (SEQ ID NO:52) | |
| GGCCAACATGGCGAAACCCGTCTCTACTAAAA | 47 |
| (SEQ ID NO:53) | |
| GGCCAACATGGCGAAA | 17, 111 |
| (SEQ ID NO:54) | |
| CCCGTCTCTACTAAAA | 138, 143 |
| (SEQ ID NO:55) | |
| GTGGAGCTTGCAGTGAGCCGAGATCGCGCCAC | 1180 |
| (SEQ ID NO:56) | |
| GTGGAGCTTGCAGTGA | 14, 992 |
| (SEQ ID NO:57) | |
| GCCGAGATCGCGCCAC | 20, 593 |
| (SEQ ID NO:58) | |

The key notion that two 16-mer tags can be aligned against the genomic database to identify a unique 32-mer coding sequence was further tested in silico in the following analysis. Using the set of 13,904 Unique RefSeq known mRNAs, two consecutive 16-mer tags were extracted near the 5′ end of 1,000 mRNAs. These 16-mer tags were then pooled into a single “bin” to mimic a tag library. We then asked whether we could successfully recover, first, the tandem tags, and, second, the correct coding region by aligning the individual 16-mer tags against the human genome database. The 32 bp result set of tandem genome alignments was compared to the original 1,000 32 bp known mRNA tandem. The results are summarized in TABLE 5 below.

Approximately 75% of the 32-mer sequences could be recovered by the tandem method. The remaining 25% not found in the genome are most likely due to the gaps and incomplete sequences present in the current version of the human genome database. The false positives, which appear because two 16-mer tags paired up illegitimately, constituted about 2%.

TABLE 5
In silico validation of the tandem tag method

| | mRNA | mRNA | 32-MER | DISTINCT | 32-MER | GENOME |
|---|---|---|---|---|---|---|
| TEST | 32-MER | 16-MER | GENOME | 32-MER | mRNAS | FALSE |
| # | SET | SET | ALIGNMENTS | TANDEMS | FOUND | POSITIVES |
| 1 | 1000 | 2000 | 35,874 | 727 | 720 | 7 |
| | (995 | (1988 | | | (720/995 = | (7/727 = |
| | distinct) | distinct) | | | 72.4%) | 0.96%) |
| 2 | 1000 | 2000 | 5,513 | 746 | 728 | 18 |
| | (991 | (1982 | | | (728/991 = | (18/746 = |
| | distinct) | distinct) | | | 73.5%) | 2.41%) |
| 3 | 1000 | 2000 | 154,854 | 758 | 752 | 6 |
| | (993 | (1981 | | | (752/993 = | (6/758 = |
| | distinct) | distinct) | | | 75.7%) | 0.79%) |
| 4 | 1000 | 2000 | 175,420 | 778 | 770 | 8 |
| | (992 | (1981 | | | (770/992 = | (8/778 = |
| | distinct) | distinct) | | | 77.6%) | 1.03%) |
| 5 | 1000 | 2000 | 910 | 736 | 729 | 7 |
| | (990 | (1979 | | | (729/990 = | (7/736 = |
| | distinct) | distinct) | | | 73.6%) | 0.95%) |
| 6 | 1000 | 2000 | 2,642 | 759 | 739 | 20 |
| | (992 | (1984 | | | (739/992 = | (20/759 = |
| | distinct) | distinct) | | | 74.5%) | 2.64%) |
| 7 | 1000 | 2000 | 1,436 | 735 | 730 | 5 |
| | (991 | (1982 | | | (730/991 = | (5/735 = |
| | distinct) | distinct) | | | 73.6%) | 0.68%) |
| 8 | 1000 | 2000 | 184,449 | 753 | 742 | 11 |
| | (992 | (1983 | | | (742/992 = | (11/753 = |
| | distinct) | distinct) | | | 74.8%) | 1.46%) |
| AVG | 992 | 1983 | | 749 | 74.5% | 1.365% |
| 1-8 | distinct | distinct | | | | |
| | sets | tags | | | | |
| 9 | 3000 | 6000 | 177,607 | 2266 | 2212 | 54 |
| | (2960 | (5913 | | | (2212/2960 = | (54/2266 = |
| | dist.) | distinct) | | | 4.7%) | 2.38%) |

Tags once extracted from the sequenced concatemers are usually subjected to a clustering protocol to positively match the tags to known transcripts or to the human genome. This is done due to the redundant occurrence of some of the 16 base pair tags within the genome, which does not allow the mining novel gene transcripts. Since the first set of tags and their tandem tags are generated from undefined ends of double-stranded cDNAs, each transcript is highly likely to generate multiple overlapping or closely spaced tags. Also, the number of such tags per transcript should be proportional to the relative abundance of the transcript in the sample. By aligning all tags against mRNA database and/or against the human genome, a stretch of physical sequence of the corresponding transcript is identified.

An example of a clustering protocol is shown below. Prior to clustering, 16 bp tags were extracted from sequenced concatemers and aligned to FASTA files of human genome, mRNA, and EST sequence databases. The output from this alignment program yields an alignment table for each respective sequence database. Each row in the alignment table is an exact location where one of the tags was found in the sequence database (GenBank Accession, strand, sequence position).

Using the genome or mRNA alignment table, tag hits are clustered by scanning each sequence (genome contig or mRNA) to group tags that are proximal to each other. The clustering program accepts two criteria: maximum hit-to-hit distance and minimum number of tag hits needed to define a cluster. The program picks up the first tag alignment and places it into the cluster bin. It continues down the genome strand until it finds the next alignment. If its distance away from the last alignment placed in the cluster bin is less than the maximum hit-to-hit distance then it is placed in the cluster bin. Clustering is finished when the next hit is too far away or the program finishes scanning the genome contig strand. If the number of hits in the cluster bin are at least the minimum number set by the user, then a cluster is created and the program outputs to a table the cluster location and other relevant information. With an mRNA alignment table, the cluster program works exactly the same way except that it scans down each mRNA instead of a genomic contig.

To ensure high quality clusters, in this example, a maximum hit-to-hit distance of no greater than the tag length (hits must be adjacent or overlapping) was used. Minimum cluster size was 3 hits.

### TAG CLUSTER EXAMPLES

1) Clustering Against mRNA Transcript Database (Refseq+Genome Annotation mRNAs)

| CLUST | | BEGIN | END | NUM |
|---|---|---|---|---|
| ID | GENBGI | POS | POS | TAGS |
| 1 | 4501858 | 1821 | 1846 | 6 |

mRNA ID:

>gi|4501858|ref|NM_{—}001609.1| Homo sapiens acyl-Coenzyme A dehydrogenase, short/branched chain (ACADSB), nuclear gene encoding mitochondrial protein, mRNA (2682 bp)

Location of transcript in Genome:

NT_{—}008926.7|17472331 PLUS strand

64789-64929 (1003-1143)

66802-66906 (1142-1246)

67437-68879 (1243-2682)

NT_{—}027097.4 PLUS strand

1770323-1770376 (4-57)

1795662-1795822 (57-217)

1799051-1799154 (215-318)

*matching genome cluster should be:

8040 (1821-1846).

Clustering against Human Genome database:

| | | | | | NUM |
|---|---|---|---|---|---|
| CLUSTID | GENBGI | STRAND | BEGINPOS | ENDPOS | TAGS |
| 3411961 | 17472331 | PLUS | 68015 | 68040 | 6 |

This corresponds with expected cluster location and size.

2) mRNA Cluster(s):

| CLUSTID | GENBGI | BEGINPOS | ENDPOS | NUMTAGS |
|---|---|---|---|---|
| 2 | 4502010 | 1364 | 1396 | 8 |
| 3 | 4502010 | 1533 | 1562 | 7 |
| 4 | 4502010 | 1587 | 1623 | 8 |

>gi|4502010|ref|NM_{—}000476.1| Homo sapiens adenylate kinase 1 (AK1), mRNA (2271 bp)

mRNA matches Genome:

NT_{—}029366.3|17449540 MINUS strand

1803682-1803643 ( 1-40)

1800671-1800631 ( 41-81)

1799083-1799043 ( 80-120)

1798874-1798709 (117-282)

1797960-1797843 (281-398)

1794533-1794339 (398-592)

*1794098-1792410 (589-2271)

*matching genome clusters should be:

1793291-1793323 (1396-1364)

1793125-1793150 (1562-1533)

1793064-1793100 (1623-1587)

Genome Cluster(s):

| | | | | | NUM- |
|---|---|---|---|---|---|
| CLUSTID | GENBGI | STRAND | BEGINPOS | ENDPOS | TAGS |
| 1862419 | 17449540 | MINUS | 1793062 | 1793098 | 8 |
| 1862420 | 17449540 | MINUS | 1793124 | 1793153 | 7 |
| 1862422 | 17449540 | MINUS | 1793289 | 1793321 | 8 |

3) mRNA Cluster(s):

| CLUSTID | GENBGI | BEGINPOS | ENDPOS | NUMTAGS |
|---|---|---|---|---|
| 5 | 4502042 | 1927 | 1959 | 9 |
| 6 | 4502042 | 2010 | 2047 | 6 |
| 7 | 4502042 | 2058 | 2131 | 12 |

>gi|4502041|ref|NM_{—}000694.1| Homo sapiens aldehyde dehydrogenase 3 family, member B1 (ALDH3B1), mRNA (2790 bp)

mRNA matches Genome:

NT_{—}009840.7|17472907 PLUS strand

1472982-1473028 (1-47)

1477929-1478094 (44-209)

1481160-1481272 (208-321)

1481406-1481528 (320-442)

1481798-1481889 (436-527)

1482346-1482431 (525-610)

1484116-1484504 (607-996)

1485227-1485398 (996-1167)

1488638-1488743 (1160-1265)

*1490381-1491906 (1263-2790)

*matching genome cluster(s) should be:

1491045-1491077 (1927-1959)

1491128-1491165 (2010-2047)

1491176-1491249 (2058-2131)

Genome Cluster(s):

| | | | | | NUM- |
|---|---|---|---|---|---|
| CLUSTID | GENBGI | STRAND | BEGINPOS | ENDPOS | TAGS |
| 3473301 | 17472907 | PLUS | 1491044 | 1491076 | 9 |
| 3473302 | 17472907 | PLUS | 1491127 | 1491164 | 6 |
| 3473303 | 17472907 | PLUS | 1491175 | 1491248 | 12 |

4)mRNA Cluster(s):

| CLUSTID | GENBGI | BEGINPOS | ENDPOS | NUMTAGS |
|---|---|---|---|---|
| 7012 | 14786455 | 2347 | 2385 | 12 |

>gi|14786455|ref|XM_{—}009672.4| Homo sapiens phosphoenolpyruvate carboxykinase 1 (soluble) (PCK1), mRNA (2642 letters)

mRNA matches Genome:

NT_{—}011362.7|17484369 PLUS strand

21189036-21189118 (1-83)

21189283-21189548 (80-345)

21189983-21190167 (345-529)

21190607-21190812 (526-731)

21190941-21191128 (732-919)

21191447-21191642 (919-1084)

21192080-21192307 (1081-1308)

21192394-21192529 (1307-1442)

21192952-21193049 (1439-1536)

21193261-21194369 (1534-2642)

*matching genome cluster(s) should be:

21194074-21194112 (2347-2385)

Genome Cluster(s):

| | | | | | NUM- |
|---|---|---|---|---|---|
| CLUSTID | GENBGI | STRAND | BEGINPOS | ENDPOS | TAGS |
| 4332399 | 17484369 | PLUS | 21194074 | 21194112 | 12 |

5) mRNA Cluster(s):

| CLUSTID | GENBGI | BEGINPOS | ENDPOS | NUMTAGS |
|---|---|---|---|---|
| 647 | 5174710 | 1385 | 1410 | 5 |
| 648 | 5174710 | 1446 | 1484 | 10 |

×gi|5174710|ref|NM_{—}005992.1| Homo sapiens T-box 1 (TBX1), transcript variant B, mRNA (1538 bp)

mRNA matches Genome:

NT_{—}011519.9|17484914 PLUS strand

2892106-2892148 (1-43)

2894958-2895080 (41-163)

2896306-2896684 (162-540)

2898641-2898747 (537-643)

2899557-2899729 (641-813)

2900361-2900516 (814-969)

2901160-2901229 (969-1038)

2901304-2901406 (1037-1139)

2918314-2918438 (1137-1261)

*2918714-2918996 (1256-1538)

*matching genome cluster(s) should be:

2918843-2918868 (1385-1410)

2918904-2918942 (1446-1484)

Genome Cluster(s):

| | | | | | NUM- |
|---|---|---|---|---|---|
| CLUSTID | GENBGI | STRAND | BEGINPOS | ENDPOS | TAGS |
| 4343636 | 17484914 | PLUS | 2918843 | 2918868 | 5 |
| 4343637 | 17484914 | PLUS | 2918904 | 2918942 | 10 |

Occasionally, alignment of two tandem 16-mer tags on the human genome produced false 32-mer sequences that probably do not exist in real transcripts. These represent a false-pairing against the human genome and are false-positives. Such false pairing can be reduced by using a second 5′ adaptor containing two degenerate nucleotide bases. This example is shown below:

Bpm I digestion

| | |
|---|---|
| 5′ . . . C T G G A G (N)16\hat{ } . . . 3′ | (SEQ ID NO:59) |
| 3′ . . . G A C C T C (N)14\hat{ } . . . 5′ | |

The first adaptor:

| | |
|---|---|
| GCAGTGGTATCAACGCAGAGTCCACGCGTCTGGAG | (SEQ ID NO:3) |
| \|\|\|\|\|\|\|\|\|\|\|\|\|\|\|\|\|\|\|\|\|\|\|\|\|\|\|\|\|\|\|\| | |
| CACCATAGTTGCGTCTCAGGTGCGCAGACCTCₚ | |

The second adaptor with 2 nn on the 3′ end of the first strand:

(SEQ ID NO:60)

GCAGTGGTATCAACGCAGAGTCCACGCGTCTGGAGNN
||||||||||||||||||||||||||||||||||
CACCATAGTTGCGTCTCAGGTGCGCAGACCTC_{P}

Bpm I digestion leaves 3′-overhang of two nucleotides on the bottom strands of the leftover cDNA to which the second adaptor with two nn 3′ overhang on the top strand is ligated. These two nucleotides are conserved in the second tag after second Bpm I cut. Hence the last two nucleotides of the first tag and the first two nucleotides of the ‘putative’ tandem tag are the same. This prevents the random matching of all the available tags to the first tag and decreases significantly the artificial combination between two random 16 mers.

TABLE 6 below lists other type II restriction enzymes that generate short DNA fragments away from the recognition sites and could be used in this method.

TABLE 6: Type II Restriction Enzymes With Asymmetric Recognition Sequences:

Type II Restriction Enzymes

Cuts after 4n Ear I, Sap I,

Cuts after 5n Alw I, Bmr I, Bsa I, BsmA I, BsmB I, MlyI, PleI,

Cuts after 6n Bbs I, BciV I, Fau I,

Cuts after 7n Mnl I,

Cuts after 8n Aar I, BfuA I, BspM I, Hph I, Mbo II, SspD5I, Sth132I,

Cuts after 9n SfaN I,

Cuts after 10n BseR I, BspCN I, Hga I,

Cuts after 11n AceIII, Eci I, TaqII, Tth111II,

Cuts after 12n Bbv I, RleAI,

Cuts after 13n BcefI, Fok I

Cuts after 14n BceA I, BsmF I, StsI,

Cuts after 16n Bce83I, Bpm I, Bsg I, Eco57I, Eco57MI,

Cuts after 20n MmeI

While the invention has been described in connection with what is presently considered to be the most practical and preferred embodiments, it is to be understood that the invention is not limited to the disclosed embodiments, but on the contrary is intended to cover various modifications and equivalent arrangements included within the spirit and scope of the appended claims.

Thus, it is to be understood that variations in the present invention can be made without departing from the novel aspects of this invention as defined in the claims. All patents and articles cited herein are hereby incorporated by reference in their entirety and relied upon.

What is claimed is:

1. A method for generating five prime biased tandem tag libraries of cDNAs, comprising the steps of:
a) isolating a sample of mRNAs;
b) synthesizing double-stranded cDNAs from the mRNAs;
c) blunt-ending the double-stranded cDNAs;
d) attaching an adapter molecule to the blunt ends of the double stranded cDNAs to form a complex,
wherein the adapter molecule is a double stranded, synthetic oligonucleotide comprising:
1) a recognition site for a type IIS restriction enzyme,
2) a cloning site for releasing tags to a cloning vector, and
3) a PCR primer site;
e) digesting the complex with a type IIS restriction enzyme to form released tags;
f) separating the released tags from the double-stranded cDNAs;
g) amplifying the released tags to form amplified tags;
h) isolating the amplified tags;
i) concatenating the amplified tags to form concatenated tags;
j) amplifying the concatenated tags; and
k) isolating the concatenated tags.

2. The method of claim 1, wherein the type IIS restriction enzyme is selected from the group consisting of Ear I, Sap I, Alw I, Bmr I, Bsa I, BsmA I, BsmB I, Mly I, Ple I, Bbs I, BciV I, Fau I, Mnl I, Aar I, BfuA I, BspM I, Hph I, Mbo II, SspD5 I, Sth132 I, SfaN I, BseR I, BspCN I, Hga I, AceIII, Eci I, TaqII, Tth111II, Bbv I, RleAI, BcefI, Fok I, BceA I, BsmF I, StsI, Bce83I, BpmI, Bsg I, Eco57I, Eco57MI, and MmeI.

3. The method of claim 1, wherein the type IIS restriction enzyme is BpmI.

4. The method of claim 1, wherein the mRNAs are from a mammal.

5. The method of claim 4, wherein the mRNAs are from a human.

6. The method of claim 1, wherein the released tags are comprised of 50 nucleotides or less.

7. The method of claim 1, wherein the released tags are comprised of 36 nucleotides or less.

8. The method of claim 1, wherein the released tags are comprised of 32 nucleotides or less.

9. The method of claim 1, wherein the released tags are comprised of at least 20 nucleotides.

10. The method of claim 1, further comprising sequencing the isolated concatenated tags to obtain a nucleotide sequence and comparing the nucleotide sequence to a known nucleotide sequence.

11. A method for generating five prime biased tandem tag libraries of cDNAs, comprising the steps of:
d) isolating a sample of mRNAs;
e) synthesizing double-stranded cDNAs from the mRNAs;
f) blunt-ending the double-stranded cDNAs;
d) attaching a first adapter molecule to the blunt ends of the double stranded cDNAs to form a first complex,
wherein the first adapter molecule is a double stranded, synthetic oligonucleotide comprising:
1) a recognition site for a type IIS restriction enzyme,
2) a cloning site for releasing tags to a cloning vector, and
3) a PCR primer site;
e) digesting the first complex with a type IIS restriction enzyme to form first released tags;
f) separating the first released tags from the double-stranded cDNAs and attaching a second adapter molecule to the double-stranded cDNAs to form a second complex;
g) amplifying the first released tags to form first amplified tags;
h) isolating the first amplified tags;
i) concatenating the first amplified tags to form first concatenated tags;
j) amplifying the first concatenated tags;
k) isolating the first concatenated tags;
l) digesting the second complex with a type IIS restriction enzyme to form second released tags;
m) separating the second released tags from the double-stranded cDNAs;
n) amplifying the second released tags to form second amplified tags;
o) isolating the second amplified tags;
p) concatenating the second amplified tags to form second concatenated tags;
q) amplifying the second concatenated tags; and
r) isolating the second concatenated tags.

12. The method of claim 11, wherein the type IIS restriction enzyme is selected from the group consisting of Ear I, Sap I, Alw I, Bmr I, Bsa I, BsmA I, BsmB I, Mly I, Ple I, Bbs I, BciV I, Fau I, Mnl I, Aar I, BfuA I, BspM I, Hph I, Mbo II, SspD5 I, Sth132 I, SfaN I, BseR I, BspCN I, Hga I, AceIII, Eci I, TaqII, Tth111II, Bbv I, RleAI, BcefI, Fok I, BceA I, BsmF I, StsI, Bce83I, BpmI, Bsg I, Eco57I, Eco57MI, and MmeI.

13. The method of claim 11, wherein the type IIS restriction enzyme is BpmI.

14. The method of claim 11, wherein the mRNAs are from a mammal.

15. The method of claim 14, wherein the mRNAs are from a human.

16. The method of claim 11, wherein the first or second released tags are comprised of 50 nucleotides or less.

17. The method of claim 11, wherein the first or second released tags are comprised of 36 nucleotides or less.

18. The method of claim 11, wherein the first or second released tags are comprised of 32 nucleotides or less.

19. The method of claim 11, wherein the first or second released tags are comprised of at least 20 nucleotides.

20. The method of claim 11, further comprising sequencing the first and second isolated concatenated tags to obtain nucleotide sequences and comparing the nucleotide sequences to a known nucleotide sequence.


SEQUENCE LISTING (60 sequences)

SEQ ID NO: 1 - DNA, 45 bp, Artificial Sequence
  Description of Artificial Sequence Synthetic oligonucleotide
gcagtggtatcaacgcagagtccagtgtggtggacgcgtctggag

SEQ ID NO: 2 - DNA, 42 bp, Artificial Sequence
  Description of Artificial Sequence Synthetic oligonucleotide
ctccagacgcgtccaccacactggactctgcgttgataccac

SEQ ID NO: 3 - DNA, 45 bp, Artificial Sequence; identical to SEQ ID NO: 1
  Description of Artificial Sequence Synthetic oligonucleotide

SEQ ID NO: 4 - DNA, 19 bp, Artificial Sequence
  Description of Artificial Sequence Synthetic oligonucleotide
acgcgtgtcgacctcgagt

SEQ ID NO: 5 - DNA, 26 bp, Artificial Sequence
  Description of Artificial Sequence Synthetic oligonucleotide
tctagactcgaggtcgacacgcgtnn

SEQ ID NO: 6 - DNA, 19 bp, Artificial Sequence
  Description of Artificial Sequence Synthetic oligonucleotide
acgcgtgtcgacctcgagt

SEQ ID NO: 7 - DNA, 22 bp, Artificial Sequence
  Description of Artificial Sequence Synthetic oligonucleotide
tctagactcgaggtcgacacgc

SEQ ID NO: 8 - DNA, 23 bp, Artificial Sequence
  Description of Artificial Sequence Synthetic oligonucleotide
gcagtggtatcaacgcagagtcc

SEQ ID NO: 9 - DNA, 16 bp, Homo sapiens
gcgcggtgtggtggca

SEQ ID NO: 10 - DNA, 16 bp, Homo sapiens
gcaggcgcagcccagc

SEQ ID NO: 11 - DNA, 16 bp, Homo sapiens
gatagatcgccatcat

SEQ ID NO: 12 - DNA, 16 bp, Homo sapiens
gaacgacaccgtaact

SEQ ID NO: 13 - DNA, 16 bp, Homo sapiens
tagatcgccatcatga

SEQ ID NO: 14 - DNA, 16 bp, Homo sapiens
acgacaccgtaactat

SEQ ID NO: 15 - DNA, 16 bp, Homo sapiens
ctgcggtggagccgcc

SEQ ID NO: 16 - DNA, 16 bp, Homo sapiens
accaaaatgcagattt

SEQ ID NO: 17 - DNA, 16 bp, Homo sapiens
gtggagctgtcgccat

SEQ ID NO: 18 - DNA, 16 bp, Homo sapiens
gaaggtcgagctgtgc

SEQ ID NO: 19 - DNA, 16 bp, Homo sapiens
gccatcgtggtgtgtt

SEQ ID NO: 20 - DNA, 16 bp, Homo sapiens
cttgactccgctgctc

SEQ ID NO: 21 - DNA, 16 bp, Homo sapiens
cagcaccatggcggtt

SEQ ID NO: 22 - DNA, 16 bp, Homo sapiens
ggcaagaacaagcgcc

SEQ ID NO: 23 - DNA, 16 bp, Homo sapiens
cttgaacctgggaggc

SEQ ID NO: 24 - DNA, 16 bp, Homo sapiens
ggaggttgcagtgaac

SEQ ID NO: 25 - DNA, 16 bp, Homo sapiens
cttgaacccaggaggt

SEQ ID NO: 26 - DNA, 16 bp, Homo sapiens
ggaggttgcagtgatc

SEQ ID NO: 27 - DNA, 16 bp, Homo sapiens
gtgtgtgtgtgtgtgt

SEQ ID NO: 28 - DNA, 16 bp, Homo sapiens
gtttgtgtgtgtgtgt

SEQ ID NO: 29 - DNA, 16 bp, Homo sapiens
ctgcggtggagccgcc

SEQ ID NO: 30 - DNA, 16 bp, Homo sapiens
accaaaatgcagattt

SEQ ID NO: 31 - DNA, 16 bp, Homo sapiens
gtggagctgtcgccat

SEQ ID NO: 32 - DNA, 16 bp, Homo sapiens
gaaggtcgagctgtgc

SEQ ID NO: 33 - DNA, 16 bp, Homo sapiens
gccatcgtggtgtgtt

SEQ ID NO: 34 - DNA, 16 bp, Homo sapiens
cttgactccgctgctc

SEQ ID NO: 35 - DNA, 16 bp, Homo sapiens
cagcaccatggcggtt

SEQ ID NO: 36 - DNA, 16 bp, Homo sapiens
ggcaagaacaagcgcc

SEQ ID NO: 37 - DNA, 16 bp, Homo sapiens
cttgaacccaggaggt

SEQ ID NO: 38 - DNA, 16 bp, Homo sapiens
ggaggttgcagtgatc

SEQ ID NO: 39 - DNA, 16 bp, Homo sapiens
cttgaacccaggaggt

SEQ ID NO: 40 - DNA, 16 bp, Homo sapiens
tgcagtgagccaagat

SEQ ID NO: 41 - DNA, 32 bp, Homo sapiens
gcactttgggaggccggctcacgcctgtaatc

SEQ ID NO: 42 - DNA, 16 bp, Homo sapiens
gcactttgggaggccg

SEQ ID NO: 43 - DNA, 16 bp, Homo sapiens
gctcacgcctgtaatc

SEQ ID NO: 44 - DNA, 32 bp, Homo sapiens
cacgcccgtaatcccaagcactttgggaggct

SEQ ID NO: 45 - DNA, 16 bp, Homo sapiens
cacgcccgtaatccca

SEQ ID NO: 46 - DNA, 16 bp, Homo sapiens
agcactttgggaggct

SEQ ID NO: 47 - DNA, 32 bp, Homo sapiens
agcactttgggaggctgagatcgagaccatcc

SEQ ID NO: 48 - DNA, 16 bp, Homo sapiens
agcactttgggaggct

SEQ ID NO: 49 - DNA, 16 bp, Homo sapiens
gagatcgagaccatcc

SEQ ID NO: 50 - DNA, 32 bp, Homo sapiens
gcttgaacctgggaggggaggttgcagtgagc

SEQ ID NO: 51 - DNA, 16 bp, Homo sapiens
gcttgaacctgggagg

SEQ ID NO: 52 - DNA, 16 bp, Homo sapiens
ggaggttgcagtgagc

SEQ ID NO: 53 - DNA, 32 bp, Homo sapiens
ggccaacatggcgaaacccgtctctactaaaa

SEQ ID NO: 54 - DNA, 16 bp, Homo sapiens
ggccaacatggcgaaa

SEQ ID NO: 55 - DNA, 16 bp, Homo sapiens
cccgtctctactaaaa

SEQ ID NO: 56 - DNA, 32 bp, Homo sapiens
gtggagcttgcagtgagccgagatcgcgccac

SEQ ID NO: 57 - DNA, 16 bp, Homo sapiens
gtggagcttgcagtga

SEQ ID NO: 58 - DNA, 16 bp, Homo sapiens
gccgagatcgcgccac

SEQ ID NO: 59 - DNA, 22 bp, Homo sapiens
  modified_base (7)..(22): a, t, c, g, other or unknown
ctggagnnnnnnnnnnnnnnnn

SEQ ID NO: 60 - DNA, 37 bp, Artificial Sequence
  Description of Artificial Sequence Synthetic oligonucleotide
gcagtggtatcaacgcagagtccacgcgtctggagnn